(19) United States
(12) Patent Application Publication
(10) Pub. No.: US 2025/0262461 A1
(43) Pub. Date: Aug. 21, 2025

(54) ULTRASOUND NEUROMODULATION GUIDED BY ARTIFICIAL INTELLIGENCE

(71) Applicant: Sanmai Technologies, PBC, Sunnyvale, CA (US)

(72) Inventor: Christopher DAFT, Tucson, AZ (US)

(21) Appl. No.: 18/959,039

(22) Filed: Nov. 25, 2024

Related U.S. Application Data
(60) Provisional application No. 63/554,004, filed on Feb. 15, 2024.

(51) Int. Cl. A61N 7/00; G06V 10/774; G16H 20/40; G16H 30/40

(52) U.S. Cl. CPC A61N 7/00; G06V 10/774; G16H 20/40; G16H 30/40; A61N 2007/0026; A61N 2007/0078; G06V 2201/03

(57) ABSTRACT

A medical ultrasonic system employs an artificial intelligence large vision model (LVM) trained on a database of target anatomy. The system employs an ultrasound probe and a neuromodulation beamformer plus additional instrumentation, all interfaced directly or indirectly to the domain specific large vision model (DSLVM). The system is configured to operate using volume ultrasound imaging or slice ultrasound imaging of the target anatomy. Examples are described for the cranial anatomy.

### CROSS-REFERENCE TO RELATED APPLICATIONS

The present application claims the benefit of U.S. Provisional Application titled “ULTRASOUND STIMULATION GUIDED BY ARTIFICIAL INTELLIGENCE”, filed on Feb. 15, 2024, and having Ser. No. 63/554,004. The subject matter of this related application is hereby incorporated herein by reference.

### TECHNICAL FIELD

This invention relates to a medical ultrasound system that employs a domain specific large vision model (DSLVM) trained on a database of target anatomy, and in particular to a system adaptable to using either volume or slice imaging.

### BACKGROUND

In a medical ultrasound system, the critical functions of targeting and neuromodulation control have previously been performed by human operators aided by computer vision algorithms. In the present invention, new methods for performing these functions are proposed.

### BRIEF DESCRIPTION OF THE DRAWINGS

FIG. 1 illustrates training of a device specific large vision model (DSLVM) using data from an MRI database for the target anatomy.

FIG. 2 illustrates a DSLVM operating in inference mode.

FIG. 3 illustrates an ultrasound system centered on a large vision model, performing inference using a volume-scanning ultrasound probe.

FIG. 4 illustrates a slice geometry comprising an azimuth probe element having elevation angle greater than azimuth angle.

FIG. 5 illustrates an alternative ultrasound system centered on a large vision model, performing inference using a slice probe.

### DETAILED DESCRIPTION OF THE INVENTION

Two medical ultrasound systems are described, each system centered on an artificial intelligence (AI) system which performs the critical functions of targeting and neuromodulation control. Large Vision Models (LVMs), a form of AI, have evolved from Large Language Models (LLMs) and are oriented towards images rather than text. Examples of early LVMs include VGGNet, GoogleNet, and ResNet. In this application, Domain Specific Large Vision Models (DSLVMs) are directed at medical ultrasound systems.

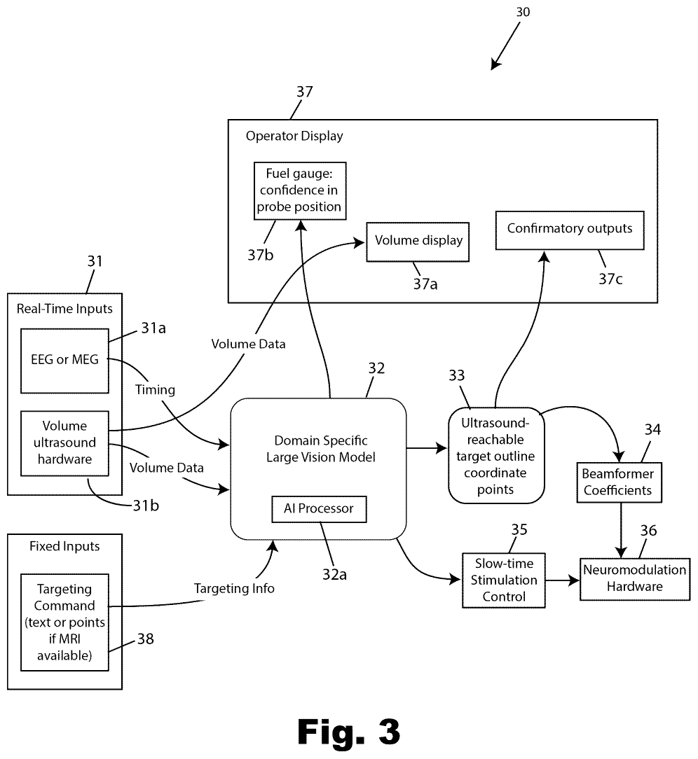

The two ultrasound systems differ in the types of probe and data acquisition systems used. The first to be described (see FIG. 3) uses a 2D or matrix probe which can acquire volume data. In it, the AI's encoded knowledge of a large database of MRI scans relates the real-time volume data being acquired by the probe to an estimated MRI. The system of FIG. 3 does not require a pre-procedure MRI scan.

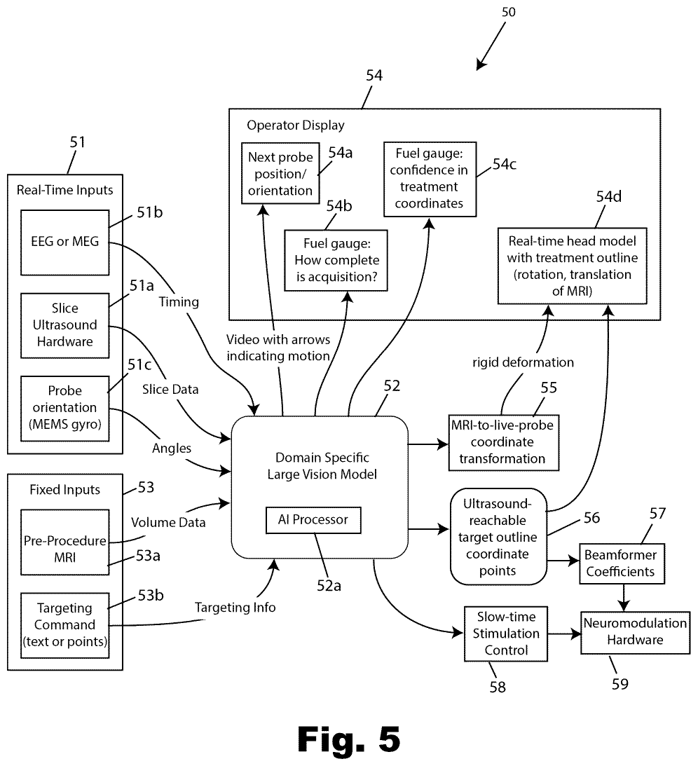

The second system (see FIG. 5) uses a 1D or other probe which produces slice data by electronically scanning in one dimension. It uses the AI to relate the real-time slice data being acquired by the probe to a pre-procedure structural MRI scan.

Three architectures have emerged for DSLVMs: attention-based, convolutional, and multi-layer perceptron. Firstly, in the prior art, the transformer architecture became a building block for constructing DSLVMs. Examples include Vision Transformer (ViT, Google Brain), Swin Transformer (Microsoft), and VideoMAE (Tencent). These DSLVMs undergo pre-training using extensive image datasets, enabling them to capture image content and extract semantic information. Meta's Segment Anything Model (SAM) operates as an image segmentation model which can be prompted.

Secondly, convolutional neural networks (CNNs) historically stood out due to their computational efficiency over multilayer perceptrons (MLPs). CNNs outperform traditional algorithms for almost all computer vision tasks, such as object detection, segmentation, de-mosaicing, super-resolution, and deblurring. The CNN architecture typically comprises alternate convolutional and pooling layers with several fully connected layers behind.

Thirdly, Google Brain developed the MLP-mixer architecture, another new computer vision paradigm. It uses neither the attention mechanism of transformers, nor convolution operations.

Disclosed herein is a DSLVM-centric ultrasound treatment system where guidance of the neuromodulation is based on real-time ultrasound plus anatomical knowledge encapsulated in a DSLVM. The target anatomy may be a structure with a subject's brain, or any other anatomy. The DSLVM may be built from any of the architectures described—a transformer, a CNN or an MLP-mixer, or another approach.

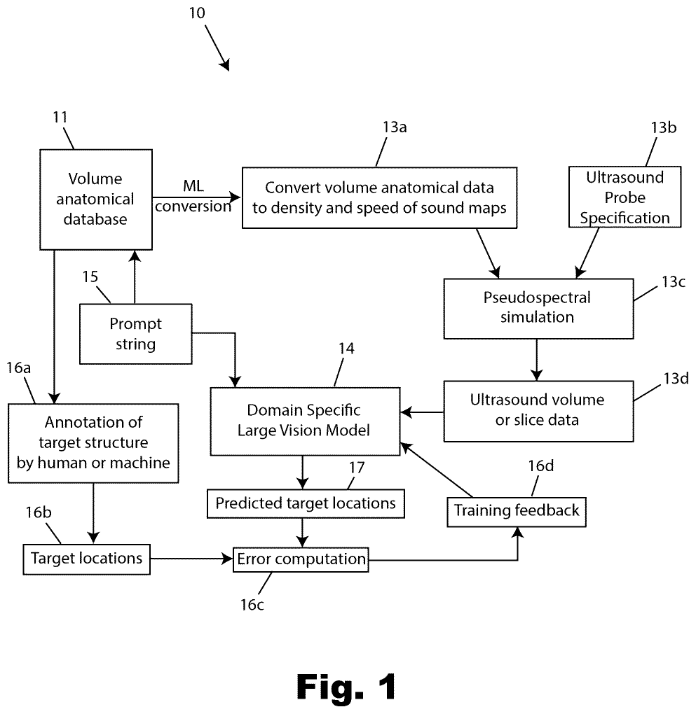

FIG. 1 illustrates a training system 10 for a DSLVM 14, employing volume anatomical database 11 to illustrate the range of anatomy in the clinical area of interest. Predicted target locations 17 corresponding to a trained DSLVM may be obtained using transfer learning from a similar model trained using MRI and computerized tomography (CT), such as V-Net, (reference 4). Alternatively, it may be trained from scratch using MRI, CT or other volumetric scans. In either case, many ultrasound training datasets are created from each MRI scan, each having a different transducer type, such as a slice probe with a 1D line of elements or a volume probe with a 2D matrix of elements. Different probe geometries and center frequencies generate further ultrasound synthetic training data from the MRI scan. Simulations for model training include many probe locations on the scalp.

The DSLVM is trained to comprehend the relationship between ultrasound slice or volume data and a volumetric map of the target anatomy. Synthetic ultrasound data indicated in the box “Ultrasound volume or slice data” 13d can be reliably obtained from MRI or other volume scanning modalities in a few steps. For example, this may be done by first creating a synthetic CT image using established deep learning methods (reference 7) and then estimating the acoustic properties 13a of the target anatomy from the synthetic CT (reference 8). Alternatively, density and speed of sound maps 13a may be computed from a database of CT scans. Other routes from volume medical imaging modalities to acoustic property data sets are also possible. With these data, a model such as a pseudospectral simulation 13c can reliably model ultrasonic propagation and scattering and create simulated ultrasonic returns to probe 13b to train the model. Transducer type and position obtained from the Ultrasound Probe Specification 13b are also input to the pseudospectral simulation 13c. Other types of ultrasonic simulation algorithms such as finite-element or finite-difference models may also be used. Labeling or visual prompting 15 may be applied to volume anatomical database 11 but this may not be required since the DSLVM may be capable of identifying the relevant structures without labels. The output of ultrasound simulation 13c, either in slice or volume form 13d, is fed to the DSLVM.

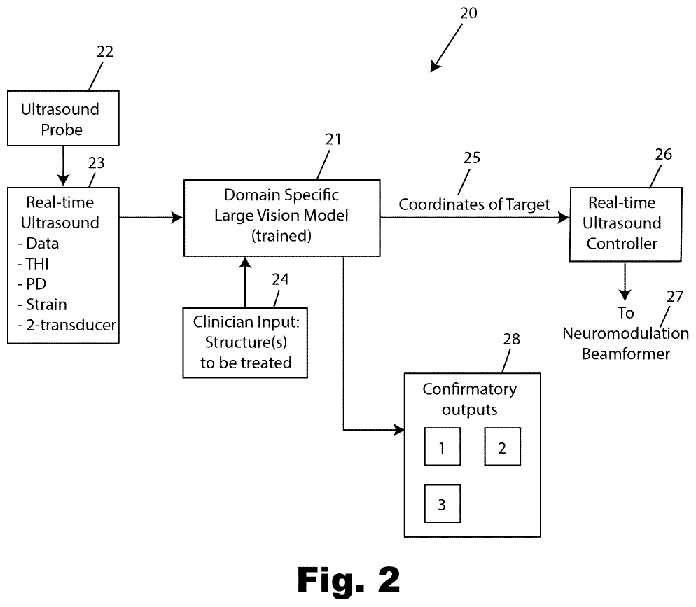

FIG. 2 illustrates a system 20 comprising a DSLVM 21 operating in

inference mode, as would be the case in a product. Ultrasound probe 22 produces real-time ultrasound data 23 in various modes which may include: tissue harmonic imaging (THI), Power Doppler (PD, blood vessels), Strain (tissue stiffness), or a 2-transducer mode that transmits using one aperture and receives using a second aperture. Real-time ultrasound data 23 may be augmented with optical or LIDAR data about the subject's head geometry from a camera swept around the target anatomy (not shown). The operator or clinician specifies the name of the structure to be treated 24 and the DSLVM outputs target coordinates 25 to Real-time ultrasound controller 26 which, after computation of beam formation coefficients, drives the Neuromodulation Beamformer 27. Calculating the beam formation coefficients of an ultrasonic field which matches coordinates bounding the target structure(s) is a well-studied area in therapeutic ultrasound (reference 9).

DSLVM 21 also creates confirmatory outputs 28. These show the operator aspects of the treatment such as a real-time anatomical illustration with the treatment volume(s) highlighted. The DSLVM is trained to produce an MRI-style anatomical estimate in the ultrasound probe's coordinate system from ultrasound data acquired at any position or angle.

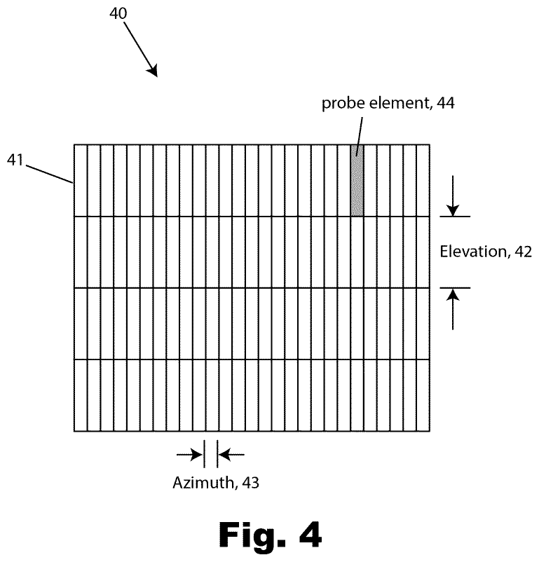

Using the background described in reference to FIGS. 1 and 2, further details are provided in each of two embodiments of the present disclosure: (i) the volume ultrasound approach and (ii) the slice ultrasound approach. The volume ultrasound approach to be described in reference to FIG. 3 is superior; it employs a probe comprising a 2D matrix of transducer elements and an application-specific integrated circuit (ASIC) to process the high information rate from the matrix array (reference 10). The slice ultrasound approach to be described in reference to FIGS. 4 and 5 produces far less real-time anatomical data which is a limitation. But the hardware system can use traditional, low-cost transducer manufacturing, which can be an advantage in practice when making a product. It has lower hardware technical risk but relies more on the capability of the DSLVM than does the implementation of FIG. 3.

FIG. 3 illustrates the volume ultrasound embodiment 30 of the present disclosure. It shows how a volume-scanning matrix probe 31b and EEG or MEG sensors 31a provide real-time inputs 31 to the DSLVM. The ultrasound volume data prompts the model 32, whose outputs 33 can be used to program a delay and amplitude controlling beamformer to provide ultrasonic neuromodulation to a target anatomy. An AI processor 32a within the domain specific large vision model 32 produces ultrasound reachable target outline coordinate points 33 used to create beamformer coefficients 34, and slow-time simulation control 35 derived from EEG/MEG 31a that provides timing information to the neuromodulation hardware 36. In an embodiment AI processor 32a comprises a network of GPU and CPU processors. DSLVM 32 acts like the kernel of an operating system; its function is analogous to the way the Linux kernel interacts with peripherals attached to its CPU. Here, devices analogous to peripherals in a Linux system include:
An electroencephalographic (EEG) 31a or magnetoencephalographic (MEG) array of sensors which can provide information about the response to neuromodulation of the target anatomy.
A matrix array of ultrasound transducers and beamforming electronics capable of volume acquisition 31b of anatomical and blood flow data.
Desired targeting data 38 specified by a clinician by either:
A text string such as “posterior cingulate cortex and amygdala,” or:
   A set of points marked by the clinician on a pre-procedure MRI scan, if available. These points define a treatment volume in the coordinate system of the MRI, which differs from the coordinate system of the neuromodulation transducer.
An operator display 37 provides access to several data sources informing a clinician of the progress made by the system towards reliable targeting of the neuromodulation. This display may include:
   Volume ultrasound data 37a being received from the imaging/guidance hardware.
   A “fuel gauge” 37b indicating to the clinician the DSLVM's confidence about the usefulness of the current probe location and orientation for the neuromodulation task.
   Confirmatory outputs 37c providing further data to the clinician on the overall credibility of the DSLVM's interpretation of the target anatomy revealed by the real-time ultrasound.
Neuromodulation hardware 36, comprising a probe, drive electronics creating appropriate neuromodulation drive voltages to be applied to each probe element, and a beamformer which translates a description of the volume to be treated to parameters controlling the amplitude and time delays of the voltages applied to the probe elements which deliver the treatment specified by the DSLVM.
Slow-time neuromodulation control 35, informed by the EEG or MEG information supplied to the LVM. These data may define when neuromodulation is applied relative to the activity in the target anatomy.

The behavior of the neuromodulation hardware 36 is controlled in two ways. Steering, focusing and other control of the spatial extent of the beam are accomplished by selection of beamforming coefficients 34. Temporal control of the neuromodulation to synchronize with aspects of physiological activity is achieved by the separate slow-time neuromodulation control 35 that is output from the DSLVM 32.

In an embodiment of the present disclosure a use-case for the volume ultrasound approach of FIG. 3 includes the following steps, wherein the target anatomy is the cranial region of a subject:
Specify the treatment region: this can be either:
   Part of prescription from the prescribing doctor.
   Wellness applications which may be accessible without prescription
Place the probe on the subject's skull.
The DSLVM converts the volume ultrasound data into anatomy and labels the treatment area.
If the target anatomy probe proves to be in a poor location for treatment, the operator display (in a clinical environment) or the subject's phone (during at-home use) may show how to reposition it.
When the probe location is adequate for treatment, treatment starts.

In an embodiment of the present disclosure FIG. 4 illustrates a 1.75D probe 40 comprising an array 41 of probe elements having elements comprising an elevation dimension 42 larger than the azimuth dimension 43. In an exemplary application, array 40 is used to steer and focus ultrasonic waves in a transcranial ultrasound (TUS) procedure. Four azimuth probe elements 44 are shown as one column of array 41. The 1.75D arrangement economizes on the total number of probe elements by only contemplating steering in the azimuth dimension. Efficacy of the accompanying tFUS procedure is achieved with this reduced number of elements using the system of FIG. 5. The smaller element count allows a standard cable to connect the probe array 41 to a signal processor. The probe can be built with traditional, low-cost manufacturing techniques employing bulk PZT. The size of the elements is insufficient to support steering in elevation, but the element dimensions are sufficient to perform aberration correction in elevation as well as in azimuth. The data created using probe array 41 may be called “slice data” which has been corrected for skull aberration.

In an embodiment of the present disclosure FIG. 5 illustrates the slice ultrasound approach 50 wherein the target anatomy is a subject's cranial region. A 1.75D probe 40 is used as a real-time input 51, connected to slice ultrasound hardware 51a, and then to DSLVM 52. The DSLVM may also receive EEG data 51b (or magnetoencephalogram data, MEG data), and probe orientation data (produced, for example, by MEMs gyro 51c. Fixed inputs 53 include pre-procedure MRI 53a (which is volume data) and a targeting command (specified as text or as a set of points marked by the operator on the MRI) 53b. An AI processor 52a within the DSLVM 52 outputs to operator display 54 the next probe position/orientation 54a, data for a “fuel gauge” showing how complete the acquisition 54b is, and a second fuel gauge showing a confidence level in the treatment coordinates 54c, and a real-time head model with treatment outline 54d. In an embodiment AI processor 52a comprises a network of GPU and CPU processors. The head model can be thought of as a rotation and translation of the pre-procedure MRI data. The real-time head model 54d is overlaid with treatment outline data from coordinate points 56. Beamformer coefficients define the steering and focusing of neuromodulation hardware 59, and are produced from coordinate points 56. Slow-time simulation control 58, for example to coordinate the timing of neuromodulation beams with physiological events sensed by EEG or MEG 51b, may also control neuromodulation hardware 59. Coordinate points may include fiducial structures or blood vessel locations within the target outline.

In an embodiment of the present disclosure a use-case for the slice ultrasound approach depicted in FIG. 5, wherein the target anatomy is a subject's cranial region, includes the following steps:
The DSLVM reads the pre-procedure MRI volume.
The operator defines the structure to be treated either as text or by annotating the MRI volume.
The operator starts to scan the subject's head with the ultrasound probe
The operator moves the probe around on the subject's head, acquiring slices for DSLVM to use. This is necessary because probe 40 is not acquiring volume data, in contrast to the situation in the system of FIG. 3.
The operator views the MRI 54d translated into the probe's coordinate system on display 54, wherein the DSLVM produces an MRI-to-live-probe coordinate transformation.
   The operator monitors the completeness of data acquired so far by observing fuel gauge 54b. as they move the probe on the subject's head.
   The DSLVM 52 provides directions 54a informing the operator of the best way to move the probe to complete the data acquisition.
Once fuel gauge 54b indicates that enough data has been acquired, probe position indicator 54a shows the ideal position for treatment.

The operator fixes the probe at the indicated position and reviews the fuel gauge 54c to decide whether the acquired data and probe position provide confidence in the treatment setup.
If the display data is approved by the clinical operator, they start the treatment

Several differences are apparent between FIGS. 3 and 5, reflecting the increased complexity in achieving full guidance control with a guidance array capable only of producing electronically scanned slice data. Specifically:
An additional real-time input may be provided to the DSLVM by a MEMS gyro 51c which reports the orientation of the probe. Similarly, a MEMS accelerometer (not shown) may also be mounted on the probe to report its motion.
Key to the operation of the apparatus of FIG. 5 is the pre-procedure MRI 53a of the subject's head. This provides per-subject anatomical information to correlate with the real-time ultrasound slice data.
The operator display includes a real-time head model with a treatment outline 54d. The DSLVM creates the treatment volume outline from a textual prompt or from a set of points marked on the MRI by the clinician. The DSLVM also determines how to rotate and translate the MRI to appear in the coordinate system of the neuromodulation transducer. This operation is simplified if the neuromodulation probe is the same as, or is rigidly attached to, the guidance probe.
As will be clear from the previously described method, the slice transducer may need to image the head from multiple viewpoints before the DSLVM has enough confidence that the probe position, and orientation can be moved to a suitable plane for neuromodulation of the target.
Another part of the display 54a shows the head outline and the location of the probe on it, together with arrows recommending translation and rotation for a new acquisition which efficiently provides an adequate concept of the brain's volume anatomy for targeting to succeed.
Another display element 54b shows the DSLVM's view of the completeness of acquisition. With a slice acquisition, several views will need to be obtained before the DSLVM can have confidence in mapping the pre-procedure MRI to the live ultrasound.
Once enough slices have been acquired for reliable targeting, the DSLVM specifies to the operator the optimal position of the probe for neuromodulation treatment. Again, arrows are helpful in showing the operator how to move from an initial location to the best location and orientation.

Various embodiments of the present disclosure are described in the following clauses. Although the following clauses describe some embodiments of the present disclosure, other embodiments of the present disclosure are also set forth above.

1. In some embodiments, a system for training a large vision model comprises a specification for an ultrasound probe, a database of volumetric anatomical scans, a prompt string defining an anatomical target, at least one computing device, and an application executed by the at least one computing device that, when executed, causes the at least one computing device to at least train a large vision model where the inputs comprise the prompt string and data obtained by simulating the field produced by the specified ultrasonic probe and one of the volumetric anatomical scans, and the trained output produces weights that generate a set of target locations.

2. The system of clause 1 wherein the specified ultrasonic probe comprises a two-dimensional array of elements.

3. The system of clauses 1 or 2 wherein the specified ultrasonic probe comprises a one-dimensional array of elements.

4. The system of any of clauses 1-3 wherein the target anatomy is a structure within a human brain.

5. The system of any of clauses 1-3 wherein the large vision model is trained on a database of cranial data.

6. In some embodiments, a system comprises an ultrasonic probe, a neuromodulation beamformer coupled to the ultrasonic probe, at least one computing device, and an application executed by the at least one computing device that, when executed, causes the at least one computing device to at least effect inference using an AI processor within a large vision model (LVM) based on a database of target anatomy and data obtained by at least one of the ultrasonic probe or the neuromodulation beamformer, and produce coordinates of the target anatomy.

7. The system of clause 6 wherein the ultrasonic probe comprises a two-dimensional array of elements.

8. The system of claim 6 wherein the ultrasonic probe comprises a one-dimensional array of elements.

9. The system of claim 6 further comprising a prompt to indicate the target structure.

10. The system of claim 6 further comprising a volume scan of the head acquired prior to the procedure.

11. The system of claim 6 further comprising beam formation electronics for neuromodulation.

12. The system of claim 6 wherein each generator comprises a rectangular element having its elevation larger than its azimuth.

13. The system of claim 6 wherein the large vision model identifies a best match out of a database of MRI volumes.

14. The system of claim 6 wherein items are displayed to a clinician confirming correct targeting.

15. The system of claim 6 wherein the large vision model is guided by ultrasonically measured coordinates of at least one of fiducial structures or blood vessel locations within the target outline.

16. The system of any of clauses 6-15, and a second fuel gauge that shows a targeting confidence level.

17. The system of any of clauses 6-16, the user interface comprising at least one of a Grayscale B mode, a Tissue Harmonic Imaging (THI) mode, a color flow mapping mode, a power doppler mode, or a strain imaging mode.

18. The system of claim 6 wherein the user interface comprises additional data provided using a body scan produced by a camera or LIDAR device.

19. The system of claim 6 wherein the target anatomy is a structure within a human brain.

20. The system of claim 6 wherein the timing of neuromodulation emissions is controlled by a slow-time modulation control provided by the LVM.

While the preceding is directed to embodiments of the present disclosure, other and further embodiments of the disclosure may be devised without departing from the basic scope thereof, and the scope thereof is determined by the claims that follow.

What is claimed is:

1. A system for training a large vision model comprising:
a specification for an ultrasound probe;
a database of volumetric anatomical scans;
a prompt string defining an anatomical target;
at least one computing device; and
an application executed by the at least one computing device that, when executed, causes the at least one computing device to at least:
train a large vision model where the inputs comprise the prompt string and data obtained by simulating the field produced by the specified ultrasonic probe and one of the volumetric anatomical scans; and
the trained output produces weights that generate a set of target locations.

2. The system of claim 1 wherein the specified ultrasonic probe comprises a two-dimensional array of elements.

3. The system of claim 1 wherein the specified ultrasonic probe comprises a one-dimensional array of elements.

4. The system of claim 1 wherein the target anatomy is a structure within a human brain.

5. The system of claim 1 wherein the large vision model is trained on a database of cranial data.

6. A system comprising:
an ultrasonic probe;
a neuromodulation beamformer coupled to the ultrasonic probe;
at least one computing device; and
an application executed by the at least one computing device that, when executed, causes the at least one computing device to at least:
effect inference using an AI processor within a large vision model (LVM) based on a database of target anatomy and data obtained by at least one of the ultrasonic probe or the neuromodulation beamformer; and
produce coordinates of the target anatomy.

7. The system of claim 6 wherein the ultrasonic probe comprises a two-dimensional array of elements.

8. The system of claim 6 wherein the ultrasonic probe comprises a one-dimensional array of elements.

9. The system of claim 6 further comprising a prompt to indicate the target structure.

10. The system of claim 6 further comprising a volume scan of the head acquired prior to the procedure.

11. The system of claim 6 further comprising beam formation electronics for neuromodulation.

12. The system of claim 6 wherein each generator comprises a rectangular element having its elevation larger than its azimuth.

13. The system of claim 6 wherein the large vision model identifies a best match out of a database of MRI volumes.

14. The system of claim 6 wherein items are displayed to a clinician confirming correct targeting.

15. The system of claim 6 wherein the large vision model is guided by ultrasonically measured coordinates of at least one of fiducial structures or blood vessel locations within the target outline.

16. The system of claim 6 wherein the user interface further comprises a first fuel gauge that shows the completeness of acquisition of anatomical information, and a second fuel gauge that shows a targeting confidence level.

17. The system of claim 6 wherein the application generates a user interface, the user interface comprising at least one of a Grayscale B mode, a Tissue Harmonic Imaging (THI) mode, a color flow mapping mode, a power doppler mode, or a strain imaging mode.

18. The system of claim 6 wherein the user interface comprises additional data provided using a body scan produced by a camera or LIDAR device.

19. The system of claim 6 wherein the target anatomy is a structure within a human brain.

20. The system of claim 6 wherein the timing of neuromodulation emissions is controlled by a slow-time modulation control provided by the LVM.
